(19) United States
(12) Patent Application Publication
(10) Pub. No.: US 2010/0168690 A1
(43) Pub. Date: Jul. 1, 2010

(54) NASAL APPLICATOR FOR A NASAL FLUID ASPIRATION DEVICE AND NASAL FLUID ASPIRATION DEVICE INCLUDING SAID NASAL APPLICATOR

(75) Inventor: Jose Bensoussan, Los Angeles, CA (US)

Correspondence Address: CANTOR COLBURN, LLP, 20 Church Street, 22nd Floor, Hartford, CT, 06103, US

(73) Assignee: UBIMED L.L.C., Miami, FL (US)

(21) Appl. No.: 12/522,941

(22) PCT Filed: Nov. 26, 2007
(86) PCT No.: PCT/FR2007/001936

§ 371 Date: Mar. 4, 2010

(30) Foreign Application Priority Data
Dec. 1, 2006 (FR) 0610516

(51) Int. Cl. A61M 1/00

(52) U.S. Cl. 604/319

(57) ABSTRACT

The invention relates to a nasal applicator (2) having a channel (3) which opens at: an opening (4) at a first end (5) of the applicator, which is intended to be inserted into a person's nose; and an opening (6) at a second end (7) of the applicator, which is provided with means (27, 28, 30) for securing same in an opening in the body of a nasal fluid aspiration device in a removable, sealed manner. The opening (6) at the second end is sealed by a non-porous element (8) which can deform elastically when a vacuum is created by a vacuum source in the body of the aspiration device and which is intended to form a nasal fluid container. The nasal applicator includes non-return means (9, 13) which prevent the nasal applicator from being emptied when a vacuum is no longer being applied to the deformable element. The elastically-deformable nasal applicator and the non-return means can deform when pressure is exerted on the nasal applicator so that the applicator and the container can be emptied.

### BACKGROUND OF THE INVENTION

The present invention relates to a nasal applicator for a nasal fluid aspiration device and a nasal fluid aspiration device including said nasal applicator.

### DESCRIPTION OF THE PRIOR ART

The document FR 2877579 describes a nasal fluid aspiration device comprising a hollow body delimiting a vacuum chamber that can be linked to a vacuum source, the hollow body comprising an opening level with the vacuum chamber. The aspiration device also comprises a nasal applicator comprising a channel opening out in an opening situated at a first end of the nasal applicator which is intended to be inserted into an individual's nose, and in an opening situated at a second end of the applicator which is equipped with means of fixing the nasal fluid aspiration device in the opening of the body in a sealed and removable manner. The opening of the second end of the applicator is blocked by a non-porous element which is elastically deformable under the effect of a vacuum created in the body of the aspiration device by a vacuum source and intended to form a container for the nasal fluids.

Thus, under the effect of a vacuum created in the vacuum chamber, a vacuum is created in the nasal applicator by deformation of the elastically deformable non-porous element. This vacuum in the nasal applicator ensures the aspiration of the nasal fluids when the first end of the applicator is inserted into an individual's nose.

The nasal applicator also comprises non-return means preventing the nasal applicator from being emptied when a vacuum ceases to be exerted on the elastically deformable element.

Without these non-return means, the pressure exerted by the elastically deformable element, which tends to resume its initial form, would cause the nasal applicator to be emptied, possibly contaminating the user of the aspiration device or resulting in a return of the fluids into the individual's nose.

One drawback of this type of aspiration device is that each nasal applicator is a single-usage applicator because of the presence of these non-return means which prevent any desirable emptying of the nasal applicator.

Thus, in the context of one and the same blowing session, there is no possibility of reusing the same nasal applicator to clean the opposite nasal fossa.

Similarly, in the event of a false maneuver caused, for example, by a movement of the person whose nose is to be blown (in particular if it is a baby or a young child), the nasal applicator becomes unusable despite the fact that it has aspirated only air.

The present invention aims to remedy these drawbacks.

The technical problem on which the invention is based therefore consists in a nasal applicator for a nasal fluid aspiration device that can be emptied whenever the user wants.

### SUMMARY OF THE INVENTION

To this end, the present invention relates to a nasal applicator for a nasal fluid aspiration device of the type described previously, which nasal applicator is elastically deformable and wherein the non-return means are deformable when a pressure is exerted on the nasal applicator so as to enable the nasal applicator and the container intended to receive the fluids to be emptied as desired.

Thus, it is possible to empty the nasal applicator by exerting a pressure on the latter. Because of this, the nasal applicator can be reused to clean the opposite nasal fossa in the context of one and the same blowing session.

Furthermore, should only air be aspirated following a misuse of the nasal fluid aspiration device, it is possible to empty the nasal applicator and the container, and reuse them.

The present invention therefore makes it possible to avoid scrapping a large number of nasal applicators and containers.

Advantageously, the non-return means comprise an elastically deformable body fixed in a sealed manner in the channel of the nasal applicator and comprising a piercing forming a part of the free section of the channel, the piercing of the body being in fluidic communication upstream with the opening of the first end of the nasal applicator and downstream with the opening of the second end of the nasal applicator.

According to another feature of the invention, the non-return means comprise a non-return valve bearing on a valve seat, the non-return valve being able to move between a channel blocking position and a channel non-blocking position under the effect of a vacuum created in the body of the aspiration device, the valve/valve seat assembly being able to be displaced and/or deformed between a channel blocking position and a position for emptying the nasal applicator under the effect of a pressure exerted on the nasal applicator.

Preferably, the non-return valve is provided roughly level with the end of the body facing the first end of the nasal applicator, the valve seat being formed by an internal shoulder provided on the walls of the channel.

According to yet another feature of the invention, the elastically deformable body is force-fitted in a complementary recess provided in the nasal applicator.

Preferably, the element blocking the second end of the nasal applicator comprises an elastically deformable impermeable membrane, such as made of latex or of polyvinyl chloride.

Advantageously, the impermeable membrane is fixed to the nasal applicator by pinching between the elastically deformable body and the nasal applicator.

According to another feature of the invention, the impermeable membrane comprises a peripheral bead intended to be held in a recess formed between the body and the nasal applicator.

The present invention also relates to a nasal fluid aspiration device comprising a hollow body delimiting a vacuum chamber that can be linked to a vacuum source, the hollow body comprising an opening level with the vacuum chamber, which device comprises a nasal applicator as claimed in one of claims 1 to 8 intended to be fixed in the opening of the hollow body.

Preferably, the hollow body contains an electric pump, driven by an electric motor, intended to create a vacuum in the vacuum chamber.

### BRIEF DESCRIPTION OF THE DRAWINGS

In any case, the invention will be clearly understood with the help of the following description, given with reference to the indexed diagrammatic drawing representing, by way of non-limiting examples, two embodiments of this nasal applicator.

FIG. 1 is a longitudinal cross-sectional view of a nasal applicator according to a first embodiment.

FIG. 2 is a front view of the non-return means in different operating positions.

FIG. 3 is a longitudinal cross-sectional view of a nasal fluid aspiration device comprising a nasal applicator according to FIG. 1.

FIG. 4 is a longitudinal cross-sectional view of a nasal applicator according to a second embodiment.

### DESCRIPTION OF THE PREFERRED EMBODIMENTS

FIG. 1 represents an elastically deformable nasal applicator 2 for a nasal fluid aspiration device.

The nasal applicator comprises a channel 3 opening out in an opening 4 situated at a first end 5 of the nasal applicator which is intended to be inserted into an individual's nose, and in an opening 6 situated at a second end 7 of the applicator which is equipped with means of fixing a nasal fluid aspiration device in an opening of the body in a sealed and removable manner.

The opening 6 of the second end is blocked by an impermeable membrane 8 which is elastically deformable under the effect of a vacuum created in the body of the aspiration device by a vacuum source and intended to form a container for the nasal fluids.

The nasal applicator 2 comprises non-return means preventing the nasal applicator from being emptied when a vacuum ceases to be exerted on the elastically deformable impermeable membrane.

The non-return means comprise a substantially cylindrical elastically deformable body 9 fixed in a sealed manner in a recess 11 provided axially in the nasal applicator and comprising a longitudinal piercing 12 forming a part of the free section of the channel 3.

The piercing 12 of the body 9 is in fluidic communication upstream with the opening 4 of the first end of the nasal applicator and downstream with the opening 6 of the second end of the nasal applicator, and therefore with the internal space delimited by the membrane 8.

The elastically deformable body 9 comprises a non-return valve 13 bearing on a valve seat 14, the valve/valve seat assembly being able to be displaced and/or deformed between a channel blocking position and a position for emptying the nasal applicator under the effect of a pressure exerted on the nasal applicator.

Furthermore, the non-return valve can move between a channel blocking position and a channel non-blocking position under the effect of a vacuum created in the body of the aspiration device. The channel non-blocking position is shown by dotted lines in FIG. 1.

The non-return valve 13 is provided roughly level with the end of the body 9 facing the first end of the nasal applicator, the valve seat 14 being formed by an internal shoulder provided on the walls of the channel.

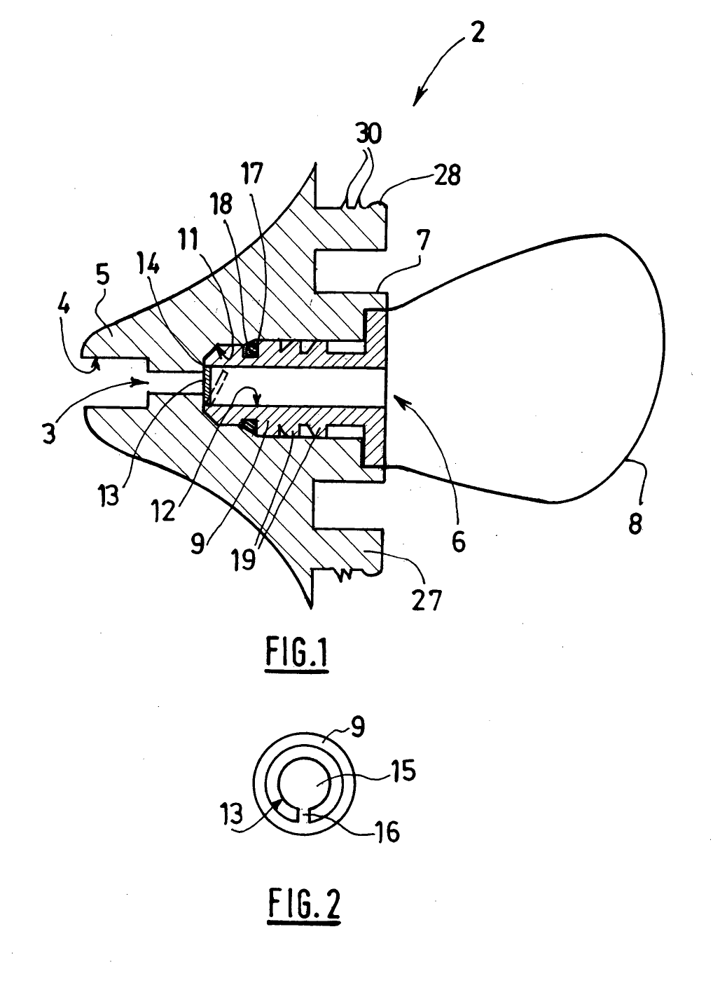

As shown more particularly in FIG. 2, the non-return valve comprises a circular flap 15 fixed to the internal wall of the body 9 by a flexible tongue 16.

It should be noted that the impermeable membrane 8 comprises a peripheral bead 17 that is intended to be retained in an annular groove 18 provided on the external surface of the body 9.

Furthermore, the body 9 comprises annular ribs 19 provided on its external surface and designed to pinch the membrane 8 against the walls of the recess 11 so as to ensure the solidity and sealing of the assembly.

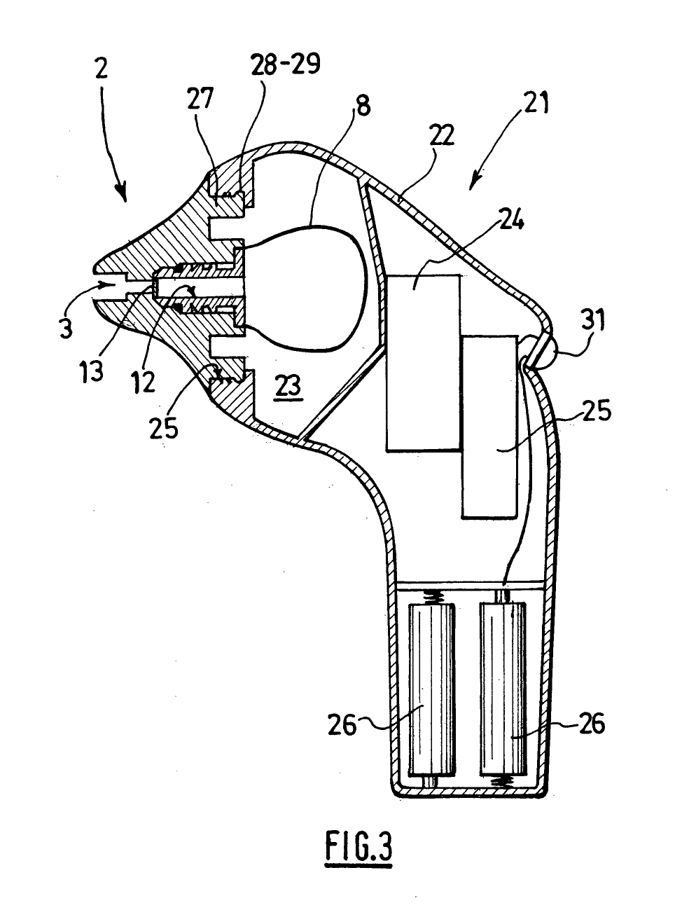

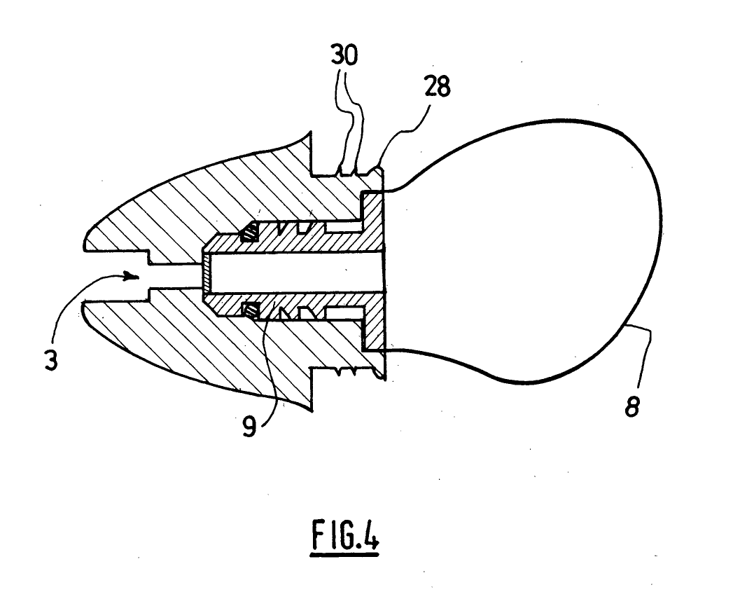

FIG. 3 represents a nasal fluid aspiration device 21 comprising a nasal applicator.

The nasal fluid aspiration device also comprises a hollow body 22 delimiting a vacuum chamber 23 linked to an electric pump 24 intended to create a vacuum in the latter. The electric pump is driven by an electric motor 25 powered by batteries 26 housed in the hollow body.

The hollow body 22 comprises, at the level of the vacuum chamber, an opening 25 intended to cooperate with the fixing means provided on the nasal applicator. These fixing means comprise an annular rib 26 provided on the external surface of a cylindrical portion 27, provided at the level of the second end of the nasal applicator. The annular rib 28 is intended to cooperate with an annular groove 29 provided at the level of the opening of the hollow body.

It should be noted that two annular ribs 30 are provided on the external surface of the cylindrical portion, these two ribs being intended to cooperate with the opening of the hollow body in order to render the fixing of the nasal applicator to the hollow body leaktight.

A pushbutton 31 is provided on the hollow body 22 in order to enable the electric motor 25 to be supplied by the batteries 26 in its pressed-in position.

Thus, when a pressure is exerted on the pushbutton, the electric pump 24 creates a vacuum in the vacuum chamber which causes the impermeable membrane 8 to be deformed.

The deformation of the impermeable membrane transmits the vacuum into the piercing 12 of the body 9. The result of this is a deformation of the non-return valve 13 which enables the nasal fluids to be aspirated.

When the vacuum ceases to be exerted on the impermeable membrane 8, the non-return valve 13 resumes bearing on the shoulder 14 and once again blocks the channel 3 under the effect of the pressure exerted by the membrane which tends to resume its initial form. The non-return valve 14 therefore makes it possible to avoid an undesired emptying of the nasal applicator and of the container intended to receive the fluids.

If the user nevertheless wants to empty the nasal applicator, all he has to do is exert a pressure on the latter, roughly at the level of the front end of the body 9. This pressure causes a deformation of the valve/valve seat assembly, so that the non-return valve no longer blocks the channel 3 of the nasal applicator. Because of this, the nasal applicator is emptied under the effect of the pressure exerted by the membrane 8.

When the emptying of the nasal applicator is finished, the membrane 8 resumes its original form and the pressure applied with the finger to the nasal applicator is stopped. The valve/valve seat assembly then resumes its original form and the non-return valve once again blocks the channel 3 of the nasal applicator. The nasal applicator can then be reused.

When the user wants to remove the used nasal applicator from the hollow body, all that he needs to do is deform the latter at the level of its second end 7 so that the annular rib 28 no longer cooperates with the groove 29 of the hollow body 22, and then withdraw the cylindrical portion 27 out of the opening 25. When the user wants to fix a new nasal applicator to the aspiration device, all that he needs to do is deform the cylindrical portion 27 of the applicator and insert the latter into the opening 22 until the rib 28 cooperates with the groove 29.

It should be noted that the structure of the valve/valve seat assembly makes it possible to systematically empty the container formed by the membrane 8, before withdrawing the nasal applicator from the aspiration device. Because of this, it is no longer necessary to provide a large-diameter opening 25 in the aspiration device to allow for the passage of a large container. Thus, it is possible to produce nasal applicators of smaller diameter, and therefore reduce their manufacturing costs and the volume of waste.

According to a variant of the invention represented in FIG. 4, the cylindrical portion 27 is eliminated and the annular ribs 28 and 30 are provided directly on the cylindrical portion 32 partly delimiting the channel 3. This structure of the applicator makes it possible to produce an opening 25 of the aspiration device that has a very small diameter.

According to another variant of the invention, the edge of the flap 15, opposite to that linked to the tongue 16, is slightly flattened in order to facilitate the emptying of the container. In this case, in order to ensure a perfect positioning of the flap 13 relative to the seat 14, the elastically deformable body 9 has an identifying mark intended to be positioned facing a complementary identification mark provided on the nasal applicator.

Needless to say, the invention is not limited to just the embodiments of this nasal applicator that are described hereinabove by way of example; it encompasses, on the contrary, all variant embodiments. Thus, in particular, the valve 13 could comprise a ball valve pushed back into its closure position by a spring.

1. A nasal applicator for a nasal fluid aspiration device comprising:
a channel which opens out in an opening situated at a first end of the nasal applicator which is intended to be inserted into an individual's nose, and in an opening situated at a second end of the applicator which is equipped with means of fixing a nasal fluid aspiration device in an opening of a body of the fluid aspiration device in a sealed and removable manner,
the opening of the second end being blocked by a non-porous element which is elastically deformable under the effect of a vacuum created in the body of the aspiration device by a vacuum source and intended to form a container for the nasal fluids,
the nasal applicator comprising non-return means preventing the nasal applicator from being emptied when a vacuum ceases to be exerted on the deformable element,
which nasal applicator is elastically deformable and wherein the non-return means are deformable under the effect of a pressure exerted on the nasal applicator so as to enable the nasal applicator and the container intended to receive the fluids to be emptied as desired.

2. The nasal applicator as claimed in claim 1, wherein the non-return means comprise an elastically deformable body fixed in a sealed manner in the channel of the nasal applicator and comprising a piercing forming a part of the free section of the channel, the piercing of the body being in fluidic communication upstream with the opening of the first end of the nasal applicator and downstream with the opening of the second end of the nasal applicator.

3. The nasal applicator as claimed in claim 1, wherein the non-return means comprise a non-return valve bearing on a valve seat, the non-return valve being able to move between a channel blocking position and a channel non-blocking position under the effect of a vacuum created in the body of the aspiration device, the valve/valve seat assembly being able to be displaced and/or deformed between a channel blocking position and a position for emptying the nasal applicator under the effect of a pressure exerted on the nasal applicator.

4. The nasal applicator as claimed in claim 3, wherein the non-return valve is provided roughly level with the end of the body facing the first end of the nasal applicator, the valve seat being formed by an internal shoulder provided on the walls of the channel.

5. The nasal applicator as claimed in claim 2, wherein the elastically deformable body is force-fitted in a complementary recess provided in the nasal applicator.

6. The nasal applicator as claimed in claim 1, wherein the element blocking the second end of the nasal applicator comprises an elastically deformable impermeable membrane, such as made of latex or of polyvinyl chloride.

7. The nasal applicator as claimed in claim 6, wherein the impermeable membrane is fixed to the nasal applicator by pinching between the elastically deformable body and the nasal applicator.

8. The nasal applicator as claimed in claim 6, wherein the impermeable membrane comprises a peripheral bead intended to be held in a recess formed between the body and the nasal applicator.

9. A nasal fluid aspiration device comprising a hollow body delimiting a vacuum chamber that can be linked to a vacuum source, the hollow body comprising an opening level with the vacuum chamber, which device comprises a nasal applicator as claimed in claim 1 intended to be fixed in the opening of the hollow body.

10. The nasal fluid aspiration device as claimed in claim 9, wherein the hollow body contains an electric pump, driven by an electric motor, intended to create a vacuum in the vacuum chamber.
